(19) United States
(12) Patent Application Publication
(10) Pub. No.: US 2004/0059248 A1
(43) Pub. Date: Mar. 25, 2004

(54) Implant for determining intra-ocular pressure

(76) Inventors: Arthur Messner, Schnaittach (DE); Tim Use, Nurnberg (DE)

Correspondence Address: MCGLEW & TUTTLE, PC, 1 SCARBOROUGH STATION PLAZA, SCARBOROUGH, NY, 10510-0827, US

(21) Appl. No.: 10/466,574

(22) PCT Filed: Jan. 10, 2002
(86) PCT No.: PCT/EP02/00160

(30) Foreign Application Priority Data
Jan. 17, 2001 (DE) 101 01 780.4

(51) Int. Cl. A61B 13/00

(52) U.S. Cl. 600/558

(57) ABSTRACT

The invention relates to an implant for determining the pressure of the aqueous humour in an eye, comprising a support body with a pressure sensor unit (5; 5″) arranged thereon, said pressure sensor unit comprising a first pressure sensor element (14; 14″) for measuring the pressure of the aqueous humour and for producing first pressure sensor data, a data processing unit (23) which is arranged on the support body and connected to the pressure sensor unit (5; 5″) in such a way that data can be transferred in order to process the first pressure sensor data and to produce first transfer data, in addition to comprising a first transmitting and receiving element which is arranged on the support body and connected to the data processing unit (23) in such a way that data can be transferred in order to transmit first transfer data and receive second transfer data from a second transmitting and receiving device arranged outside the eye.

The present invention relates to an implant for determining the pressure of the aqueous humour in an eye according to the preamble of claim 1.

All over the world, glaucoma is a major cause of blindness. The basic cause is an increased interior pressure of the eye which in most cases results from a reduced drain of aqueous humour. In order to select appropriate drugs or to suggest an operation, it is necessary to continuously record the interior eye pressure which may vary significantly during the day.

The present methods of recording the interior eye pressure in a non-invasive way use applanation tonometry. When applying this method, the cornea is deformed from outside, and the force required for this is correlated with the interior eye pressure. This method has several disadvantages: On the one hand, the result of the measurements is affected by non-adjustable and individually varying disturbances such as the rigidity of the cornea. Another drawback lies in the use of the tonometer. In most cases, only well instructed staff is capable of picking up the interior eye pressure in a discontinuous manner. The recording of series of measuring values showing the course of changes of the interior eye pressure over one or several days normally requires in-patient treatment in a hospital. This means that the course of changes of the interior eye pressure during normal daily life can hardly be determined. Furthermore, it is impossible to record the course of changes of the interior eye pressure in very short time intervals over a longer period.

DE 198 58 172 A1 teaches an intra-ocular lens which includes a measuring unit for determining the intra-ocular pressure. The measuring unit in the form of a telemetric endo-system comprises a pressure sensor element for measuring the intra-ocular pressure, a data processing unit for the production of a pressure controlled output signal and a range system with a micro coil. The micro coil serves for the receipt of feed, control and data signals and can transmit measuring and data signals. The measuring unit is disposed on a thin support film with conducting tracks which are in electric connection with the individual components. A drawback of this arrangement resides in that accurate detection of the intra-ocular pressure relative to the surroundings is not possible.

DE 197 28 069 C1 describes an implant for continuous determination of the intra-ocular pressure. The implant comprises a pressure sensor element for determination of the intra-ocular pressure, a data processing unit for conversion of the sensor signals into wirelessly transmitted information, a data logger and a transmit-receive unit. The pressure sensor element comprises a micro-mechanical pressure sensor. The measuring data continuously supplied by the pressure sensor can be stored in the data logger. Accurate determination of the intra-ocular pressure relative to the direct surroundings of the front chamber is not possible, the pressure sensor only measuring absolute pressure.

It is therefore an object of the present invention to provide an implant which allows continuous recording of the pressure of the aqueous humour.

This object is achieved by the features specified in the characterizing part of claim 1. The crux of the invention is to provide two pressure sensor elements on an implant. One pressure sensor element measures the interior pressure of the aqueous humour. A second pressure sensor element is provided, measuring the ambient pressure. The overpressure of the aqueous humour is determined by the data of the two pressure sensor elements.

Other advantageous embodiments of the invention will become apparent from the dependent claims.

Additional advantages and details of the invention will become apparent from the description of three example embodiments with reference to the attached drawings in which:

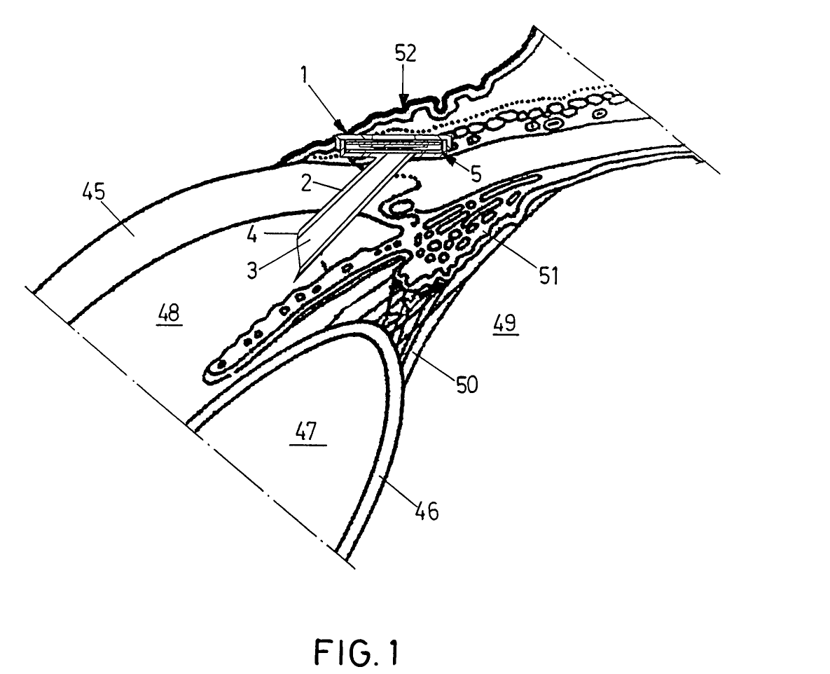

FIG. 1 shows an implant inserted into an eye according to the present invention in accordance with a first embodiment;

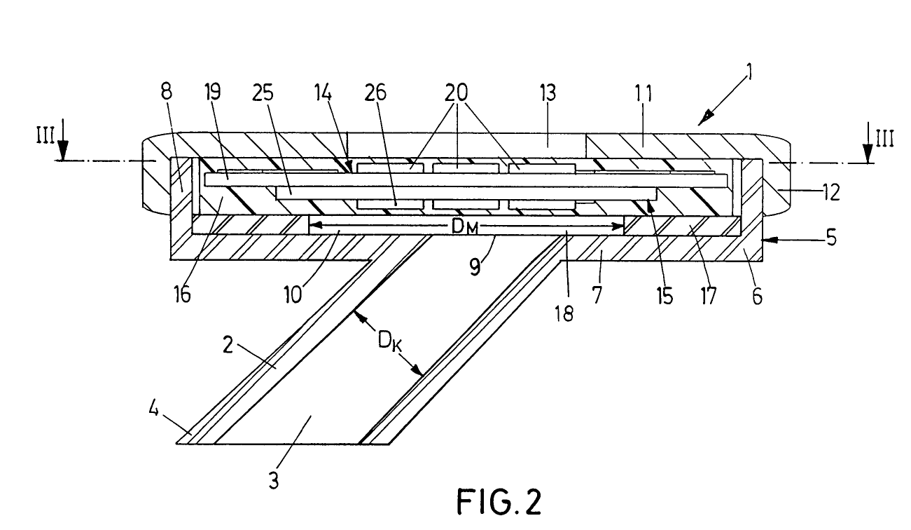

FIG. 2 shows a cross-section through the implant;

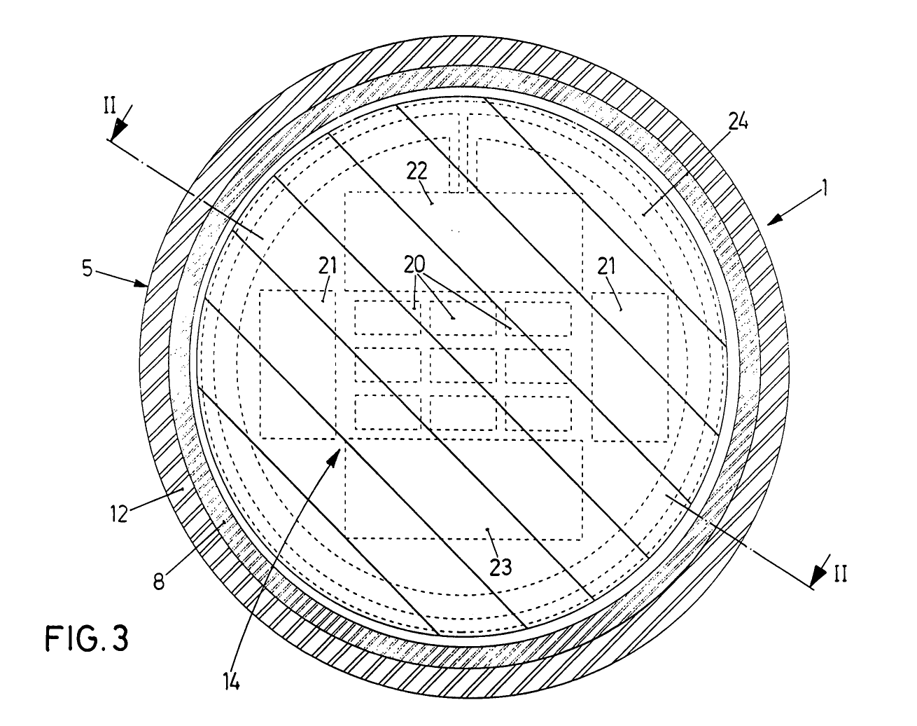

FIG. 3 shows a cross-section through the implant along the line III-III in FIG. 2;

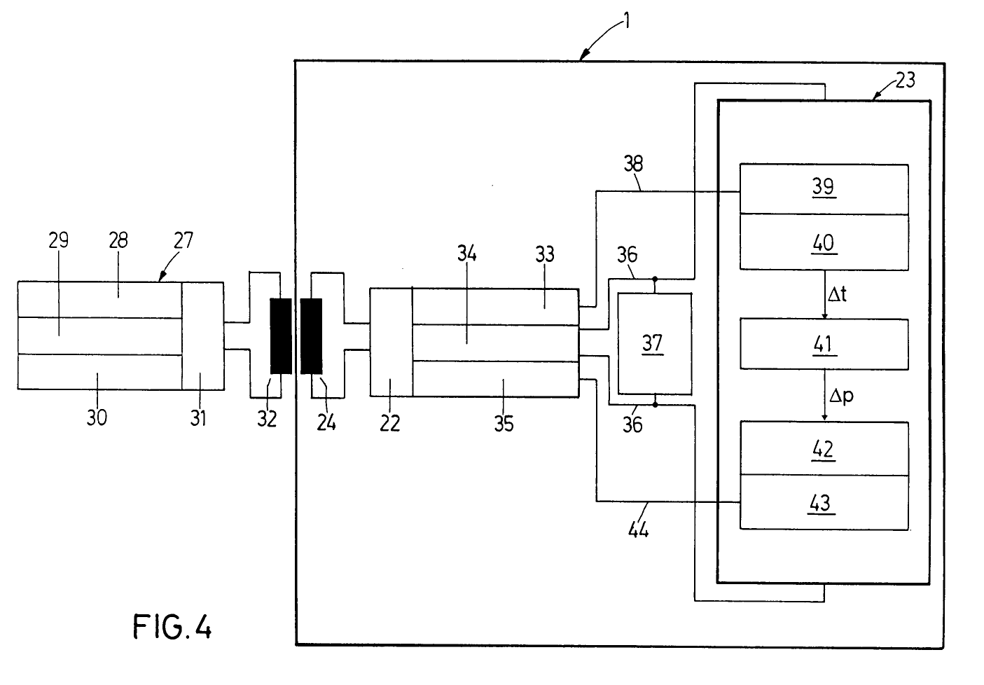

FIG. 4 shows a schematic circuit diagram of the implant illustrated in FIG. 1;

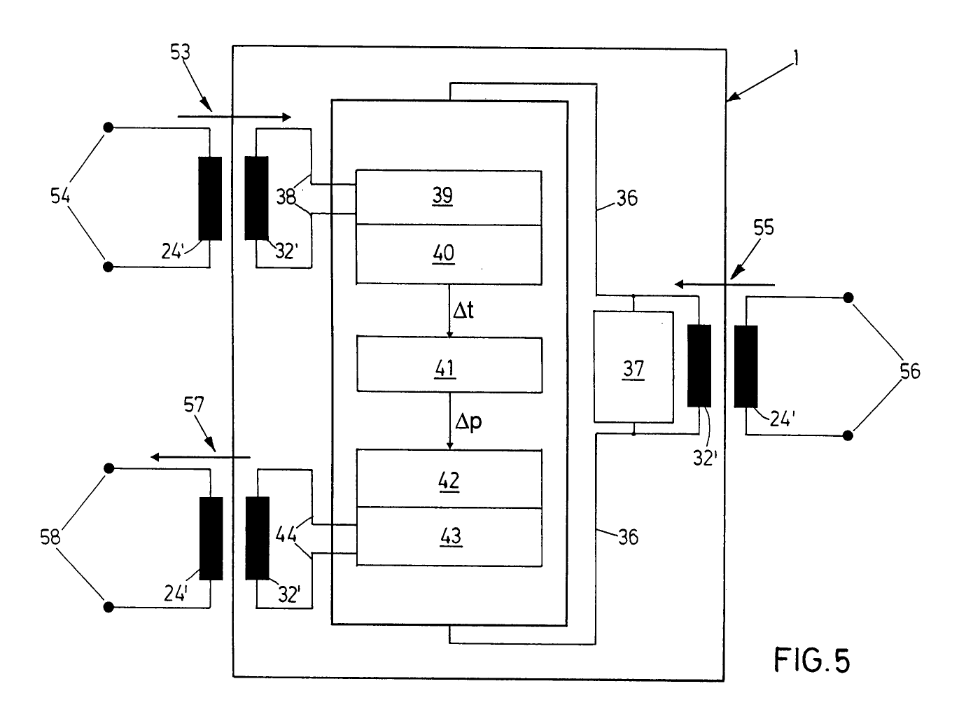

FIG. 5 shows a schematic circuit diagram of an implant in accordance with a second embodiment;

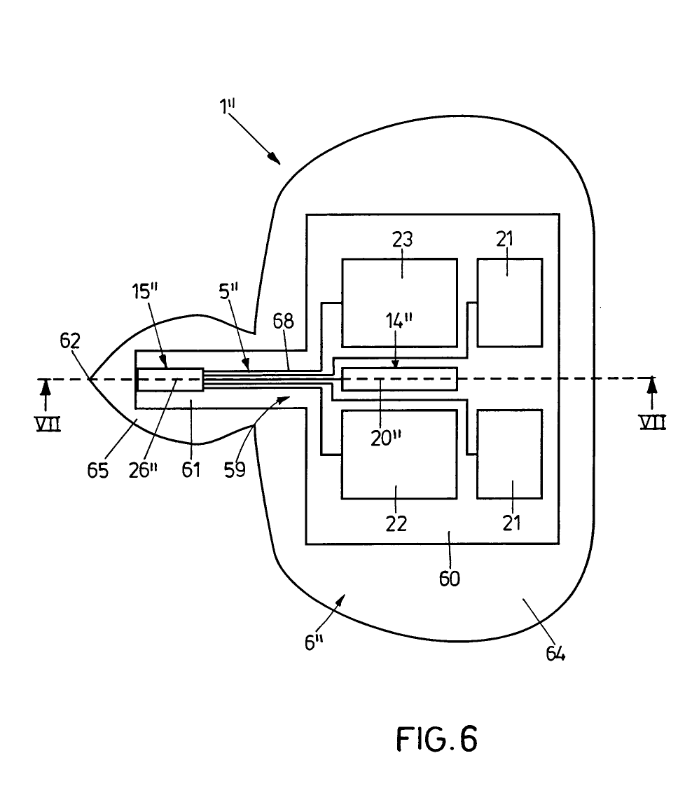

FIG. 6 shows a plane view of an implant in accordance with a third embodiment; and

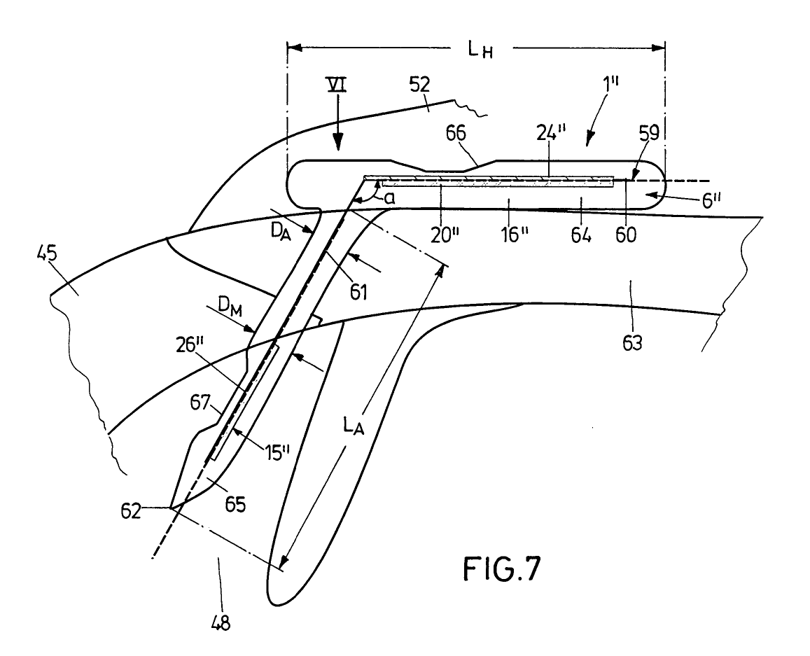

FIG. 7 shows a cross-section through the implant along the line VII-VII in FIG. 6.

Now a first embodiment of the invention will be described with reference to FIGS. 1 through 4. An implant 1 for measuring the pressure of the aqueous humour in the front ventricle of an eye comprises a hollow needle 2 enclosing a feeding channel 3. Said feeding channel 3 is open to the outside on one end 4 of said hollow needle 2. On the opposite end of said hollow needle 2, the latter is connected to a pressure sensor unit 5. Said pressure sensor unit 5 comprises a housing 6 having a circular bottom 7 formed as supporting body and an annular cylindrical wall 8 projecting upwards therefrom. Said bottom 7 comprises a central aperture 9 with said hollow needle 2 being joined with said bottom 7 along the periphery of said aperture 9. Thus, said channel 3 opens into the interior space 10 of said housing 6. Said housing 6 is closed with a top lid 11 comprising an annular cylindrical edge 12 which surrounds said wall 8 in a sealing manner and is connected with it by bonding or locking. Said lid 11 comprises a central aperture 13 through which the atmospheric pressure acts on said interior space 10. In said interior space 10, a first pressure sensor element 14 and a second pressure sensor element 15 are arranged with the second element being bonded sheet-like onto the first element and with both elements being embedded in a plastic matrix 16. Said plastic matrix 16 is essentially shaped as a flat cylinder. Said plastic matrix 16 is supported by a flat annular seal 17 on said bottom 7 of said housing 6, wherein a measuring chamber 18 is formed between said matrix 16 and said bottom 7 with said annular seal 17 forming the peripheral border of said measuring chamber 18 and with said measuring chamber 18 being connected to said channel 3. The diameter D_{M} of said measuring chamber 18 is larger than the inner diameter D_{K} of said hollow needle 2.

Now the structure of said pressure sensor elements 14 and 15 will be described in greater detail. Said pressure sensor element 14 comprises a circular substrate 19 on the centre of which an array of one or more, e.g. 3 time 3, micromechanical pressure sensors 20 is provided. These are micromechanical absolute pressure sensors common in the market which detect pressure capacitively or piezo-resistively. Two measuring memories 21, a controller 22 and a central data processing unit or CPU 23 are provided adjacent and electrically connected to said membranes 20. Along the edge of said substrate 19, an annular transmitter coil 24 is provided which ends up in said controller 22. Said membranes 20 measure the ambient pressure absolutely, i.e. compared with a known pressure present behind said membranes 20. Beneath said substrate 19, another disk-shaped substrate 25 is provided which comprises in its centre a number of sensor membranes 26 projecting downwards and designed as micromechanical absolute pressure sensors for measuring the pressure of a liquid supplied through said channel 3. As the diameter D_{M} of said measuring chamber 18 is larger than the diameter D_{K} of said channel 3, a comparatively large number of sensor membranes 26 can be arranged while said hollow needle 2 is kept as thin as possible. Said sensor membranes 26, too, measure the pressure compared with a known pressure present behind them, i.e. the absolute pressure. Said CPU 23 subtracts the absolute pressures measured by said membranes 20 and 26 from one another in order to determine the pressure of the liquid in said hollow needle 2, i.e. the overpressure of the aqueous humour, compared with the ambient pressure.

Now the data processing in said pressure sensor unit 5 and the communication with the environment will be described in greater detail with reference to FIG. 4. An external control device 27 is located outside of said implant 1 and comprises a program transmission unit 28, a power transmission unit 29, and a measuring data transmission unit 30 which units are connected via a controller 31 with a transmitter coil 32. Within said implant 1, said transmitter coil 24 allocated to said transmitter coil 32 for the telemetric transmission of data and power is connected with said controller 22 which on its part is connected with a program transmission unit 33, a power transmission unit 34, and a measuring data transmission unit 35. Said power transmission unit 34 is connected by means of lines 36 via a power-storing unit 37 to said CPU 23 to provide a power supply to the latter. Said program transmission unit 33 is connected by means of a line 38 to said measuring program memory 39 and a measurement control unit 40, which units pick up data in time intervals Δt from the sensor signal unit 41 and determine on their basis, in a downstream measuring data processing unit 42, the relative pressure Δp and store it in a measuring value memory 43 and/or 21, which memory is connected by means of a line 44 to said measuring data transmission unit 35.

Now the implantation of said implant 1 into the eye with reference to FIG. 1. The space between the cornea 45 and the iris plane is designated as front ventricle 48 which contains aqueous humour. The rear ventricle is located between the iris plane and the vitreous body 49 located behind the lens 47. The rear ventricle, too, is filled with aqueous humour. Said lens 47 is connected with the ciliary body 51 by means of zonula fibres 50. Said implant 1 is inserted into the edge or limbal portion 52 of the eye, wherein said hollow needle 2 penetrates the limbus from outside so that aqueous humour can flow from the front ventricle 48 of the eye through said channel 3 to said membranes 26. Said pressure sensor unit 5 is located outside the front chamber 48 in the episcleral tissue beneath the conjunctiva.

Now the operation of said implant 1 will be described. The aqueous humour of the front ventricle of the eye 48 flows through said channel 3 to said membranes 26. There the pressure is measured compared with a known pressure. At the same time, said membranes 20 measure the ambient pressure compared with a known pressure. Said measuring data processing unit 42 calculates from these signals the medically relevant pressure difference. Said implant 1 is a long-term implant. For power supply and data transmission, said control device 27 is placed near said implant 1. This can, for example, be accomplished by accommodating said control device 27 in spectacles. Said power transmission unit 29, said controller 31, said transmitter coils 32 and 24, said controller 22, and said power transmission unit 34 co-operate to charge said power store 37. The capacity of said power store 37 is chosen so that the power supply of the implant is guaranteed for a longer period of time and that the time interval for recharging the power store can be made as long as possible. Said program transmission unit 28, said controller 31, said transmitter coils 32 and 24, said controller 22, and said program transmission unit 33 can be used to alter said measuring program memory 39. In this manner, the time interval Δt in which the measuring values are recorded can be changed from the outside. The pressure difference determined by said measuring data processing unit 42 is stored in the measuring value memory 43. When a telemetric connection is established between said control device 27 and said implant 1, the data are transmitted from said measuring value memory 43 via said measuring value transmission unit 35, said controller 22, said transmitter coils 24 and 32, said controller 31 to said measuring data transmission unit 30 where they can be read and medically used. Said measuring value memory 43 is designed so that in case of a memory overflow the data stored first will be erased first. Should the time interval for reading be exceeded, the latest course of eye pressure changes will be retained. The use of an optical or acoustic signal transmitter makes it possible to inform the patient about a pathological increase of the interior eye pressure, so that appropriate therapeutic action can be taken without delay.

Now a second embodiment of the present invention will be described with reference to FIG. 5. Identical components are given the same reference numerals as in the first embodiment, to which reference is made here. Functionally identical but structurally differing components are given the same reference numerals followed by an inverted comma. The main difference compared with the first embodiment is that said units 33, 34, and 35 are not connected to said transmitter coil 24 via a common controller 22 but that every unit comprises a transmitter coil of its own. In the area of a first coil unit 53, the telemetric program transmission is accomplished from an external programming device 54 to said measuring program memory 39. In the area of a second coil unit 55, the telemetric power transmission is accomplished from an external power supply unit 56 to said power store 37. A third coil unit 57 accomplishes the reading of data from said measuring value memory 43 and the data transmission to an external measuring data collecting unit 58. The advantage of this arrangement is that, compared with the first embodiment, no controller 22 is required. As a drawback, a number of transmitter coils 32 are required within the implant so that it has to be larger.

Now a third embodiment of the present invention will be described with reference to FIGS. 6 and 7. Structurally identical components are given the same reference numerals as in the first embodiment, to which reference is made here. Functionally identical but structurally differing components are given the same reference numerals followed by two inverted commas. The essential difference compared with the first embodiment relates to the design of the housing 6″ and in particular to the fact that all electronic components are provided on a conducting film 59. The implant 1″ comprises as a supporting body a conducting film 59 comprising a flat, essentially rounded rectangular main portion 60 and a web-shaped front ventricle portion 61 projecting outward. Said conducting film 59 is encapsulated in a one-piece plastic housing 6″ made of biologically compatible material. Said housing 6″ comprises a main housing 64 surrounding said main portion 60 of said conducting film 59 and a housing arm 65 extending therefrom at an angle a of approximately 120° downwards and including said portion 61 of said conducting film 59, wherein said arm 65 comprises a pointed outer end 62. Said arm 65 ends in a tip at its outer end 62 in order to facilitate the pushing of said arm 65 through the sclera 63 of the eye. The length L_{H} of said main housing 64 essentially corresponds with the length L_{A} of said housing arm 65. However, other dimensions are possible as well.

Various electronic elements are formed on said conducting film 59 by means of known microtechnical structuring such as the so-called flip chip technology. On said main portion 60, said measuring value memory 21, said controller 22, said central data processing unit 23 as well as a first pressure sensor element 14″ with sensor membranes 20″ are provided. Near said elements 20″, 21, 22, and 23, the transmitter coil 24″ is placed on the opposite side of said conducting film 59. Immediately above said sensor membranes 20″, said housing 6″ comprises an area 66 having a lesser thickness. This area provided for transmitting the ambient pressure to said sensor membranes 20″ is chosen so that said membranes 20″ are sufficiently protected from the surrounding tissue and tissue fluid while, on the other hand, the ambient pressure is passed on to said membranes 20″ essentially unchanged to be measured there. It is also possible to provide the other electronic elements known from the first and second embodiment on said conducting film 59. The second pressure sensor element 15″ with its sensor membranes 26″ is provided on said portion 61 of said conducting film 59. Here, too, a pressure transmission area 67 of lesser thickness in said housing 6″ is provided adjacent to said membranes 26″ so that said membranes 26″ are one the one hand sufficiently protected from the surrounding tissue and the aqueous humour while on the other hand the interior pressure of the aqueous humour can be measured as flawlessly as possible. The pressure element 15″ and the other elements 20″, 21, 22, 23 on the main portion 60 of said conducting film 59 are connected with each other by conducting tracks 68 on said conducting film 59 to transmit data and to maintain the power supply. The length L_{A} of said housing arm 65 is chosen so that the outer third in the front ventricle of the eye 48 is immersed in aqueous humour. Said pressure sensor element 15″ is located in this outer third. Said arm 65 has in its central third a thickness D_{M} larger than the thickness D_{A} of said arm 65 in the area of said main housing 64. In this manner, said arm 65 is prevented from slipping out of said front ventricle of the eye 48.

A particular advantage of the design of the third example embodiment is the fact that all electronic elements are provided on one conducting film 59. This enables miniaturisation and mass manufacturing without problems, as known techniques of microelectronics and especially flip chip techniques may be used. Pressures in two different spaces, i.e. the front ventricle of the eye 48 and the limbal space 52, can be measured. The physiologically relevant overpressure of the aqueous humour in the front ventricle of the eye can be determined compared with the surroundings of the front ventricle of the eye.

1. An implant for determining the pressure of the aqueous humour in an eye, comprising:
a. a supporting body,
b. a pressure sensor unit (5; 5″) arranged on said supporting body and comprising a first pressure sensor element (15; 15″) for measuring the pressure of the aqueous humour and for generating first pressure sensor data,
c. a data processing unit (23) arranged on said supporting body and connected to said pressure sensor unit (5; 5″) in a data-transmitting manner for generating first transmission data, and
d. a transmitting and receiving unit arranged on said supporting body and connected to said data processing unit (23) in a data-transmitting manner for transmitting said first transmission data to and for receiving second transmission data from a second transmitting and receiving unit arranged outside the eye, characterized in that
e. said pressure sensor unit (5; 5″) comprises a second pressure sensor element (14; 14″) for measuring the ambient pressure and for generating second pressure sensor data.

2. An implant according to claim 2, characterised in that said data processing unit is designed for processing said second pressure sensor data and for determining the overpressure of the aqueous humour compared with the environment.

3. An implant according to claim 2, characterised in that said pressure sensor elements (14, 15; 14″, 15″) comprise at least one micromechanical pressure sensor (20, 26; 20″, 26″).

4. An implant according to claim 1, characterised in that said supporting body comprises at least one hollow needle (2) having a first end (4) for receiving the aqueous humour and a second end connected to said pressure sensor unit (5).

5. An implant according to claim 5, characterised in that said pressure sensor unit (5) comprises a housing (6, 11) connected to the at least one hollow needle (2).

6. An implant according to claim 2, characterised in that said first pressure sensor element (14) and/or said second pressure sensor element (15) is/are embedded in a plastic matrix (16).

7. An implant according to any of the preceding claims, characterised in that said pressure sensor unit (5; 5″) comprises a power store.(37).

8. An implant according to claim 8, characterised in that said power store (37) can be supplied with power wirelessly from the outside.

9. An implant according to claim 1, characterised in that said central data processing unit (23) is designed so that it prompts storing of the interior pressure of the aqueous humour in predetermined time intervals Δt.

10. An implant according to claim 1, characterised in that said pressure sensor unit (5″) is provided on a conducting film (59).

11. An implant according to claim 11, characterised in that said conducting film (59) comprises a main portion (60) and a laterally and web-like projecting front ventricle portion (61).

12. An implant according to claim 12, characterised in that said first pressure sensor element (14″) is arranged on said front ventricle portion (61).
